(19) United States
(12) Patent Application Publication
(10) Pub. No.: US 2022/0096747 A1
(43) Pub. Date: Mar. 31, 2022

(54) CONTINUOUS DOSING SYSTEMS AND APPROACHES

(71) Applicant: AMGEN INC., Thousand Oaks, CA (US)

(72) Inventors: Adam B. McCullough, Westlake Village, CA (US); Paul Daniel Faucher, Escondido, CA (US); Antonio S. Murcia, Oceanside, CA (US); Nathan Thomas Balcom, Vista, CA (US); William Wistar Rhoads, Ramona, CA (US); Nicholas D.M. Prsha, Encinitas, CA (US)

(21) Appl. No.: 17/426,194

(22) PCT Filed: Feb. 11, 2020
(86) PCT No.: PCT/US20/17576

§ 371 Date: Jul. 28, 2021

Related U.S. Application Data
(60) Provisional application No. 62/804,741, filed on Feb. 12, 2019.

(51) Int. Cl. A61M 5/168; A61M 5/36; A61M 5/145; A61M 39/22

(52) U.S. Cl. CPC A61M 5/16886; A61M 5/16813; A61M 5/36; A61M 5/14586; A61M 39/223; A61M 5/165; A61M 2205/3306; A61M 2205/3317; A61M 2205/3344; A61M 2205/505; A61M 2205/18; A61M 2205/3327

(57) ABSTRACT

A drug delivery system includes a delivery container including a container body adapted to accommodate a drug therein, a supply line, and a flow rate monitor. The delivery container further includes inlet and outlet ports and is constructed from a resilient material that exerts an urging force on the drug to expel the drug from the outlet port. The supply line is operably coupled to the outlet port to deliver the drug to a user. The flow rate monitor is operably coupled to at least one of the delivery container or the supply line and includes a digital controller, a fluid valve operably coupled to the digital controller, and a diaphragm assembly in fluid communication with the fluid valve and operably coupled to the digital controller. The fluid valve, the diaphragm assembly, and the digital controller cooperate to regulate a flow rate of the drug.

### CROSS-REFERENCE TO RELATED APPLICATION

Priority is claimed to U.S. Provisional Patent Application No. 62/804,741, filed Feb. 12, 2019, the entire contents of which are incorporated herein by reference.

### FIELD OF DISCLOSURE

The present disclosure generally relates to drug delivery systems and methods. More particularly, the present disclosure relates to improved approaches for preparing and delivering dosing systems.

### BACKGROUND

Drugs are administered to treat a variety of conditions and diseases. Intravenous (“IV”) therapy is a drug dosing process that delivers drugs directly into a patient's vein using an infusion contained in a container (e.g., a pliable bag). These processes may be performed in a healthcare facility, or in some instances, at remote locations such as a patient's home. A disposable IV pump in the form of an elasticized balloon may be used in an at-home setting to provide patients the ability to administer their own dosages. These take-home systems typically lack programming, are offered in a range of volumes and flow rates, and get lighter throughout delivery without the need for expensive maintenance and/or service infrastructure. However, oftentimes drugs in these disposable systems need to stay within a specific flow rate window, but they cannot alert a patient if the device becomes blocked or otherwise occluded. Compared to reusable systems, disposable systems generally do not rely on large, bulky electronics for proper operation, rather, these devices typically use their inherent elasticity to create a drug delivery pressure that, combined with tubing resistance, results in a predetermined drug flow rate. Conversely, reusable systems oftentimes have large power supplies that enable continued use for multiple days, and typically include a user interface having multiple, complex menus. In some examples, flow rate monitors may be used to monitor and adjust fluid flow of the drug. However, these systems are typically power-hungry and can have undesirable fluid pressure accuracies during varying stages of the drug administration process.

As described in more detail below, the present disclosure sets forth systems and methods for dosing techniques embodying advantageous alternatives to existing systems and methods, and that may address one or more of the challenges or needs mentioned herein, as well as provide other benefits and advantages.

### SUMMARY

In accordance with a first aspect, a drug delivery system includes a delivery container including a container body adapted to accommodate a drug therein, a supply line, and a flow rate monitor. The delivery container further includes inlet and outlet ports and is constructed from a resilient material that exerts an urging force on the drug to expel the drug from the outlet port. The supply line is operably coupled to the outlet port to deliver the drug to a user. The flow rate monitor is operably coupled to at least one of the delivery container or the supply line and includes a digital controller, a fluid valve operably coupled to the digital controller, and a diaphragm assembly in fluid communication with the fluid valve and operably coupled to the digital controller. The fluid valve, the diaphragm assembly, and the digital controller cooperate to regulate a flow rate of the drug.

In some examples, the digital controller causes the fluid flow control device to actuate the fluid valve or valves. The fluid valve may be in the form of a primary three-way valve and a secondary three-way valve. The primary three-way valve includes a primary valve inlet, a first primary valve outlet, and a second primary valve outlet. The secondary three-way valve includes a first secondary valve inlet, a second secondary valve inlet, and a secondary valve outlet. The primary and secondary three-way valves are ganged or otherwise coupled to each-other. In other examples, the fluid valve may be in the form of a four-way valve.

In some examples, the diaphragm assembly includes a reservoir that defines an internal volume, a first side port, and a second side port. The diaphragm assembly further includes a diaphragm disposed within the internal volume of the reservoir between the first and second side ports to define first and second cavities. The diaphragm may be constructed from a resilient material.

In some examples, at least one end of travel sensor is provided that senses at least one directional limit of the diaphragm. In some of these approaches, the end of travel sensor may be in the form of an optical sensor. In other approaches, the end of travel sensor may be in the form of at least one of an electrical contact sensor or a capacitive sensor. In other examples, the end of travel sensor may be in the form of a Hall Effect sensor or a pressure monitor. Other examples are possible.

In some approaches, the flow rate monitor may further include an interface coupled to the digital controller to receive at least one input and a display coupled to the digital controller. Further, the system may include an alarm operably coupled to the digital controller, an air trap, a filter, a flow restrictor, and/or a fluid path compliance member disposed downstream of the flow digital controller.

In accordance with a first aspect, a drug delivery system includes a delivery container including a container body adapted to accommodate a drug therein, a supply line, and a flow rate monitor. The delivery container further includes inlet and outlet ports and receives a driving force that causes the container body to exert an urging force on the drug to expel the drug from the outlet port. The supply line is operably coupled to the outlet port to deliver the drug to a user. The flow rate monitor is operably coupled to at least one of the delivery container or the supply line and includes a digital controller, a fluid valve operably coupled to the digital controller, and a diaphragm assembly in fluid communication with the fluid valve and operably coupled to the digital controller. The fluid valve, the diaphragm assembly, and the digital controller cooperate to regulate a flow rate of the drug.

### BRIEF DESCRIPTION OF THE DRAWINGS

The above needs are at least partially met through provision of the continuous dosing system and approaches described in the following detailed description, particularly when studied in conjunction with the drawings, wherein:

FIG. 1 illustrates an example take-home, disposable drug delivery system in accordance with various embodiments;

FIG. 2a illustrates an example diaphragm assembly of the example take-home, disposable drug delivery system of FIG. 1 in a resting state in accordance with various embodiments;

FIG. 2b illustrates the example diaphragm assembly of the example take-home, disposable drug delivery system of FIG. 1 in a first position in accordance with various embodiments;

FIG. 2c illustrates the example diaphragm assembly of the example take-home, disposable drug delivery system of FIG. 1 as fluid begins to fill the diaphragm assembly in accordance with various embodiments;

FIG. 2d illustrates the example diaphragm assembly of the example take-home, disposable drug delivery system of FIG. 1 in a third position in accordance with various embodiments;

FIG. 3 illustrates an alternative example take-home, disposable drug delivery system in accordance with various embodiments;

FIG. 4 illustrates an example flow rate monitor having an integrated manifold inside the flow rate monitor in accordance with various embodiments;

FIGS. 5a-5c illustrate a first example end of travel sensor for an example take-home, disposable drug delivery system in accordance with various embodiments;

FIGS. 6a-6c illustrate a second example end of travel sensor for an example take-home, disposable drug delivery system in accordance with various embodiments;

FIGS. 7a-7c illustrate a third example end of travel sensor for an example take-home, disposable drug delivery system in accordance with various embodiments;

FIGS. 8a-8e illustrate a fourth example end of travel sensor for an example take-home, disposable drug delivery system in accordance with various embodiments;

FIGS. 9a-9c illustrate a fifth example end of travel sensor for an example take-home, disposable drug delivery system in accordance with various embodiments;

FIGS. 10a-10c illustrate a sixth example end of travel sensor for an example take-home, disposable drug delivery system in accordance with various embodiments;

FIGS. 11a-11c illustrate a seventh example end of travel sensor for an example take-home, disposable drug delivery system in accordance with various embodiments;

FIGS. 12a-12c illustrate a eighth example end of travel sensor for an example take-home, disposable drug delivery system in accordance with various embodiments;

FIG. 12d illustrates a pressure over time curve for the example end of travel sensor of FIGS. 12a-12c in accordance with various embodiments;

FIGS. 13a-13d illustrate an alternative example fluid valve for a take-home drug delivery system in accordance with various embodiments; and

FIGS. 14a-14c illustrate alternative example fluid valves for a take-home drug delivery system in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Turning to the figures, pursuant to these various embodiments, a disposable, take-home drug delivery system 100 is provided. The drug delivery system varies from an electromechanical programmable IV pump in that the systems such as the drug delivery system 100 described herein relies primarily and/or partially on material characteristics of the pump (as opposed to an external power source) to administer a drug to a patient. These take-home systems described herein are typically smaller, lower cost, and easier to use compared to electromechanical programmable IV pumps, and as a result, can be used in settings outside of a healthcare facility (e.g., at a patient's home, office, and/or other location). By focusing on a single therapeutic or class of therapeutics, a simpler approach to a user interface and risk assessment may be afforded, thereby potentially reducing costs of goods sold (“COGS”), power requirements, and size, thus increasing value to patients. The system 100 includes a small, energy efficient “add-on” unit that may be incorporated into a take-home pump system with minimal complexity. The system 100 may be used in intravenous, subcutaneous, intra-arterial, intramuscular, and/or epidural delivery approaches having delivery times between approximately five minutes and upwards of approximately 72 hours. By using the drug delivery system 100 described herein, patient anxiety and confusion is reduced due to the use of a positive pressure flow that eliminates the need for regulatory guidance for air bubble detection as compared to peristaltic pump mechanisms. The systems described herein provide an optional, single use, pre-programmed add-on unit that provides limited functionality at the patient level. Accordingly, the add-on system is simplified.

The system 100 includes a drug delivery container 102 (e.g., an intravenous drug delivery container) which could also be considered a medication reservoir that includes a container body 103 having an inner volume 104 that accommodates a drug 101 therein. In the illustrated example, the system 100 further includes a container 105 that surrounds the drug delivery container 102 for safety and other purposes. In some examples, the container 105 may be rigid. The inner volume 104 may be sterile. This container 102 may be an off-the shelf disposable elastomeric pump of any desired size. In the illustrated example, the delivery container 102 also functions as the drive mechanism that causes the drug 101 to be administered to the patient.

Specifically, the container body 103 may be constructed from an elastic and/or resilient material. Generally speaking, the container body 103 is in a relaxed state prior to filling the drug 101 therein, and upon inserting the drug 101 into the container body 103, the container body 103 is expanded or stretched outwardly, and the inner volume 104 increases. The elasticity of the container body 103 generates a contraction force on the inner volume 104 that ultimately is exerted on the drug 101 for drug administration. In some examples, the container body 103 may be resilient or non-resilient, but may receive a driving force exerted thereon that in turn causes the container body 103 to exert an urging force on the drug 101 for drug administration. In these examples, the driving force may come from a spring member. In other examples, the driving force may be generated by a non-resilient surface that translates generally linearly in a cylinder under pressure from a spring or other resilient member.

The container 102 further includes an inlet fill port or mechanism 106 and an outlet port or mechanism 108. These ports 106, 108 may be of any type to allow for selective coupling of drug containers, vials, syringes, and the like. In some examples, the inlet fill port 106 and the outlet fill port 108 may include a valve or sealing mechanism to selectively permit fluid flow, and may be capped to prevent external contamination. Coupled to the outlet port 108 is an IV pump supply line or tubing 110 that is operably coupled to the outlet fill port 108 and dimensioned to accommodate flow of the drug 101 for patient administration (for example, via IV needle 118). This IV supply line 110 may be an off the shelf item and may have any number of desired characteristics such as length and/or flexibility. Any number of additional components may be coupled to the IV supply line 110 such as, for example, clamps 112, clips, filters (e.g., air elimination filters or traps 114), flow restrictors 116 and the like.

Typically, healthcare professionals (e.g., clinical pharmacies) stock a variety of delivery containers 102, thereby enabling ready access to the reservoir and drive (i.e., the motive force). One such example brand of delivery containers 102 is Easy Pump (e.g., Easy Pump LT 125-5-S, LT 279-27-S, etc.) which may include inner volumes 104 varying from approximately 15 mL to approximately 500 mL. These models may be in the form of high flow, medium flow, low flow, and/or ultra-low flow, and may result in a wide array of desired drug flow rates (e.g., between approximately 0.3 mL/day and approximately 500 mL/hour). As a result, a nominal infusion time may vary between approximately 5 minutes and upwards of approximately 72 hours depending on the desired usage.

The system 100 additionally includes a flow rate monitor 120 that may be operably coupled to the IV supply line 110 to monitor a flow rate of the system 100. In some examples, the flow rate monitor 120 may be directly coupled to the outlet port 108. In some examples, the flow rate monitor 120 may be configured as a flow rate controller, and thus may include a digital controller 122, a power source 124, a fluid valve 126 operably coupled to the digital controller 122, and a diaphragm assembly 128 operably coupled to the digital controller 122 and in fluid communication with the IV supply line 110. The flow rate monitor 120 may additionally include any number of optional components such as, for example, an interface 130, an alarm 132, and a filter 134 (e.g., a 35 micron filter positioned upstream of the valve 126).

The flow rate monitor 120 may be provided with the drug delivery system 100 packaging to encourage its use (though its use is not required in the event a healthcare professional has strong preferences opposing its use). In other words, the flow rate monitor 120 may be an optional component in the take-home drug delivery system 100 that the healthcare professional and/or the patient may use as they deem appropriate. The flow rate monitor 120 may be in the form of a housing that accommodates each of the components therein, and may include an inlet port 120a and an outlet port 120b, each of which may include any number and/or types of connecting ports, and may include internal tubing 121 (or, in some examples, an internal flow channel) extending between the inlet port 120a and the outlet port 120b through which the drug 101 may pass.

The flow rate monitor 120 differs from complex electromechanical infusion pumps by lacking user/patient programmability. Specifically, the flow rate monitor 120 is “programmed” at a location that is upstream from the user's at-home environment (e.g., at a pharmacy prior to providing the patient with their prescription). In this sense, the flow rate monitor 120 may be viewed as a single-use, fixed programmed, pre-grammed or pre-programmed device that only provides the patient with a limited feature set (e.g., initiate or pause dosages). Further, compared to complex electromechanical IV pumping systems, the flow rate monitor 120 described herein additionally lacks the typical programmable features afforded to healthcare professionals. In some examples, the “programmability” afforded to healthcare professionals may be limited to simply inputting the prescribed drug and/or dosage information. Accordingly, in some examples, the flow rate monitor 120 may not be reprogrammable after an initial programming.

The digital controller 122 includes software 122a adapted to control its operation, any number of hardware elements 122b (such as, for example, a non-transitory memory module and/or processors), any number of inputs, any number of outputs, and any number of connections. The software 122a may be loaded directly onto a non-transitory memory module of the digital controller 122 in the form of a non-transitory computer readable medium, or may alternatively be located remotely from the digital controller 122 and be in communication with the digital controller 122 via any number of controlling approaches. The software 122a includes logic, commands, and/or executable program instructions which may contain logic and/or commands for controlling the flow rate monitor 120. The software 122a may or may not include an operating system, an operating environment, an application environment, and/or the user interface 130. Generally, the digital controller is adapted to cause the flow rate monitor to actuate the fluid valve or valves. The valve or valves may be solenoid driven, shape memory wire (e.g., muscle wire) driven, and/or motor driven. Other examples are possible.

The power source 124 may be any type of power source capable of powering the components in the flow rate monitor 120. For example, the power source 124 may be in the form of a single or multi-cell battery commonly used in a wrist watch dimensioned to power the flow rate monitor 120 during a complete administration cycle. In one example, 250 ml of drug 101 may be delivered over a period of four days with a bolus interval of 45 minutes. Accordingly, in this example, 128 doses of bolus will be administered at a rate of 1.953 ml per bolus. The flow rate monitor 120 may require a sensor power of 23 mAh, and a valve power of 0.7 mAh. Accordingly, a power source 124 capable of providing 75 mWh may be used. Other examples are possible.

The fluid valve 126 may be in the form of any number of three-way valves. For example, the fluid valve 126 may be in the form of a primary three-way valve 136 that includes a primary valve inlet 136a, a first primary valve outlet 136b, and a second primary valve outlet 136c. The fluid valve 126 further includes a secondary three-way valve 138 that includes a first secondary valve inlet 138a, a second secondary valve inlet 138b, and a secondary valve outlet 138c. The primary three-way valve 136 is coupled or ganged to the secondary three-way valve 138 such that selective switching of the primary three-way valve 136 also causes selective switching of the secondary three-way valve 138.

The primary valve inlet 136a selectively couples (e.g., via the digital controller 122) to one of the first primary valve inlet 136b or the second primary valve outlet 136c during operation. Further, the secondary valve outlet 138c selectively couples (e.g., via the digital controller 122 or through the ganged connection to the primary three-way valve 136) to one of the first secondary valve inlet 138a or the second secondary valve inlet 138b during operation. Generally, such operation allows the diaphragm assembly 128 to fill with drug 101 via the delivery container 102, and expel the drug out the outlet port 120b of the flow rate monitor 120.

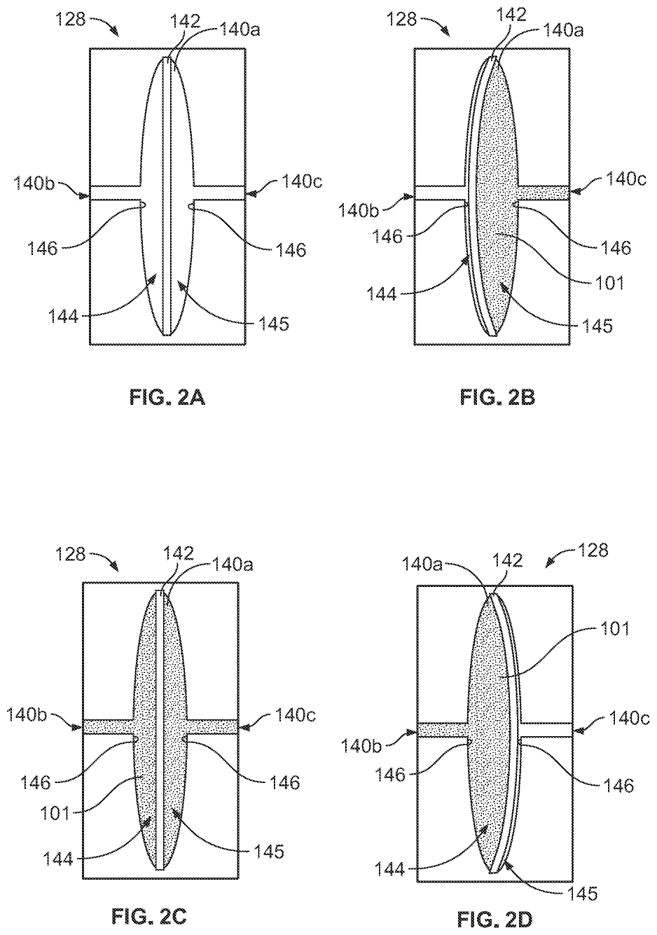

The diaphragm assembly 128 includes a reservoir 140 defining an internal volume 140a and includes a first side port 140b and a second side port 140c. The diaphragm assembly 128 further includes a diaphragm 142 which may be constructed of a resilient and/or flexible material that is disposed within the internal volume 140a between the first side port 140b and the second side port 140c. The diaphragm 142 is constrained within the internal volume 140a of the reservoir 140 so the reservoir 140, when full and empty, offer predictable delivery cycle volumes. Lower resistance is preferred to enable flat delivery rates across a wider portion of the pressure curve of the delivery container 102. As a result, the diaphragm 142, combined with the reservoir 140, defines a first cavity 144 and a second cavity 145 (see, e.g., FIGS. 2a-2d). Generally, during operation of the diaphragm assembly 128, the internal volume 140a of the reservoir 140 fills with drug 101 and, depending on which of the first side port 140b or the second side port 140c the drug 101 enters the internal volume 140a from, urges the diaphragm 142 towards the first side port 140b or the second side port 140c. As a result, the volume of the first cavity 144 and the second cavity 145 increases and decreases. The diaphragm assembly 128 further includes at least one end of travel sensor 146 that determines when the diaphragm 142 is at or against a respective sidewall of the reservoir 140.

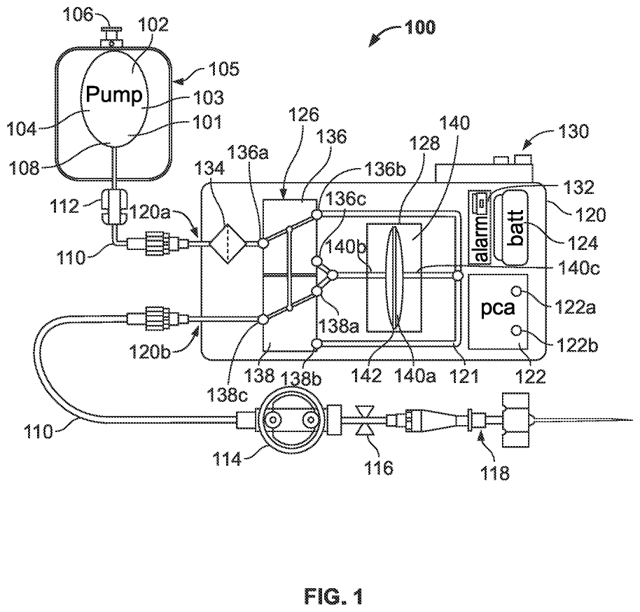

More specifically, as illustrated in FIGS. 1 and 2a-2d, in a first configuration, the pump 102 may exert a force on the drug 101 contained therein that causes the drug 101 to be expelled from the outlet port 108 and into the inlet port 120a of the flow rate monitor 120. The drug 101 enters the primary valve inlet 136a of the primary three-way valve 136, exits the first primary valve inlet 136b of the primary three-way valve 136, and flows to the second side port 140c of the reservoir 140. As illustrated in FIG. 2b, the drug 101 then pushes against the diaphragm 142 to cause the volume of the second cavity 145 to increase. As the diaphragm 142 approaches and/or contacts the sidewall of the reservoir 140, the end of travel sensor 146 senses the end of travel of the diaphragm 142 and transmits a signal to the digital controller 122. The digital controller 122 then actuates or switches the fluid valve 126 to cause the primary valve inlet 136a to be fluidly coupled to the second primary valve outlet 136c, which in turn causes the secondary valve outlet 138c to be fluidly coupled to the second secondary valve inlet 138b. In such a configuration, the drug 101 may then enter the first cavity 144 via the first side port 140b (see FIG. 2c).

As shown in FIG. 2d, the pump 101 and/or the resilience of the diaphragm 142 then causes the drug 101 contained in the second cavity 145 to be expelled therefrom via the second side port 140c. The pressure exerted by the pump 102 may be greater than a resilience of the diaphragm 142. As a result, the drug 101 contained in the second cavity 145 flows through the second secondary valve inlet 138b of the secondary three-way valve 138, through the secondary valve outlet 138c, and to the outlet port 120b of the flow rate monitor 120 to be delivered to the user. As the diaphragm 142 approaches and/or contacts the sidewall of the reservoir 140 nearest the second side port 140c, the end of travel sensor 146 senses the end of travel of the diaphragm 142 and transmits another signal to the digital controller 122. The digital controller 122 then actuates or switches the fluid valve 126 to cause the primary valve inlet 136a to again be fluidly coupled to the first primary valve inlet 136b, which in turn causes the secondary valve outlet 138c to be fluidly coupled to the secondary three-way valve 138a. At this time, the pump 102 urges the drug through the inlet port 120a of the flow rate monitor 120, the primary valve inlet 136a of the primary three-way valve 136, the first primary valve inlet 136b, and through the second side port 140c of the reservoir 140, thus urging the diaphragm 142 towards the first side port 140b. The drug contained within the first cavity 144 is in turn urged out the first side port 140b to the first secondary valve inlet 138a of the secondary three-way valve 138, and out the secondary valve outlet 138c, the outlet port 120b of the flow rate monitor 120 to be administered to the patient.

Accordingly, the combination of timing and the confirmation that the diaphragm 142 has travelled a controlled distance allows the flow rate monitor 120 to effectively act as a flow meter that uses positive displacement instead of complex fluid properties (e.g., localized micro-heating and measurement of heat change with many assumptions in an algorithm such as laminar flow, a lack of bubbles, and/or device orientation that may be incorrect). As a result, the volume per cycle may be controlled down to low pressure levels (e.g., approximately 2-3 psi). Such a system has no frictional interface, thus minimizing pressure losses in the diaphragm assembly 128.

The user interface 130 may include a number of inputs (e.g., buttons) and/or displays that allow a healthcare professional and/or a patient to initially configure the flow rate monitor 120. Generally, the interface 130 includes a limited number of patient-level settings and inputs to reduce user confusion. For example, a healthcare professional may use the interface 130 to input a desired flow rate, a duration of drug delivery, and/or a risk profile for the specific drug 101 being administered, and this input or inputs will be transmitted to the digital controller 122. Accordingly, the flow rate monitor 120 will function as a flow controller. In some examples, all or some of this information may be already stored on the digital controller 122, and thus the healthcare professional may only need to enter the drug name and/or dosage. As previously stated, the software 122a on the digital controller 122 may be capable of determining desired output values required to operate the flow rate monitor 120 based on the input or inputs received from the interface 130 and determine required tolerances (e.g., threshold and/or alarm values), in which case the flow rate monitor 120 is used to monitor flow rates. Put another way, the interface 130 may be configured to only generate an output and may not receive any inputs beyond a selection of a desired drug.

The interface 130 may additionally include buttons that begin and/or pause operation of the system 100 so that a user may begin drug administration at a desired time. The interface 130 may also include a display that can indicates desired and/or actual flow values, error messages, remaining dosage time, and the like. In some examples, the interface may be disposed on or within the flow rate monitor 120, or optionally may be implemented via external connectivity (e.g., via a portable electronic device such as a smart phone, computer, tablet, etc.).

The optional alarm 132 may function as a feedback device to alert the user of a potential problem (e.g., a full and/or partial occlusion) in the system 100. The alarm may be in the form of a speaker that produces an audible noise, a buzzer that vibrates, and/or a light that flashes. Other examples are possible. Upon the digital controller 122 receiving an input value from the user interface 130 that indicates a desired drug and/or dosage to be administered, the digital controller 122 may optionally initiate a risk profile corresponding to the selected drug. This risk profile may include an indication of an allowable flow rate range for the particular drug 101 being administered and/or any additional important operational values associated with the drug. In these examples, upon a user inputting settings (e.g., the particular drug, a desired flow rate, etc.) into the interface 130, the digital controller 122 may determine the appropriate risk profile, which can include an alarm value, via software 122a. In the event that the sensed flow value obtained from the end of travel sensors 144 exceeds this alarm value, the digital controller 122 may transmit a signal that causes the alarm 132 to be triggered and/or actuated. For example, the alarm value may be a range of approximately 10-15% from the desired flow rate. In other words, if the measured or sensed flow rate is higher or lower than 10%-15% of the desired flow rate, the alarm may be triggered, thus alerting the user to take appropriate action. Advantageously, by using the alarm 132, the patient will no long need to restart on a new delivery cycle upon occurrence of an occlusion.

In some examples, the system 100 may additionally include at least one compliance member in the form of a flexible tube, a diaphragm, and/or a bellows that predictably absorb high frequency fluid displacement operation. Some drug delivery systems operating at high frequencies (e.g., more than 50% duty cycle, or where chamber is filling for at least 50% of the time) may need a compliance member to ensure delivery accuracy. Lower frequency delivery allows sufficient time to ‘equalize’ and create predictable delivery, but for high frequencies (e.g., when using components such as a rigid flow digital controller system) there may be reduced accuracy after the system has completely primed and eliminated air bubbles (e.g., compliance). A compliance member positioned downstream of the flow digital controller may assist in ensuring delivery accuracy.

So configured, the flow rate monitor 120 may be implemented as an optional component in existing delivery systems 100 used in a variety of locations including a patient's home, office, or other non-medical facility environment. In some examples, the flow rate monitor 120 may be water resistant or waterproof to enable use while a user bathes. The flow rate monitor 120 may be provided with a coiled second supply line that automatically retracts, thus staying out of the way of the user.

Advantageously, the flow rate monitor 120 provides increased accuracy as compared to conventional reusable systems (e.g., conventional systems have an accuracy of approximately ±15%, while the system described herein may result in an accuracy of approximately ±6%) and may reduce and/or eliminate patient sensitivity to running out of drug 101. The flow rate monitor 120 may allow for a constant pressure to be delivered over longer periods of time. Further, the need to overfill the container 102 is eliminated due to less wasted medication and feedback in the case of blockage. Advantageously, alarms are minimized through the use of custom risk profile based on the specific drug 101.

The flow rate monitor 120 may be replaced at each refill interval, so battery 124 needn't occupy a large volume. Accordingly, the flow rate monitor 120 may have a small, discrete, patient-friendly size that is easy to transport and is suitable for pain management. In some examples, by pairing a relatively high flow displacement pump with the flow rate monitor 120, a low duty cycle may be provided that only allows flow for approximately 6% of the overall administration time, thereby reducing amount of time the valve 126 needs to be powered. Most drug delivery cycles may be averaged over time such that the flow rate monitor 120 delivers numerous high flow rates for short periods of time, which is the clinical equivalent to constant, low flow rates.

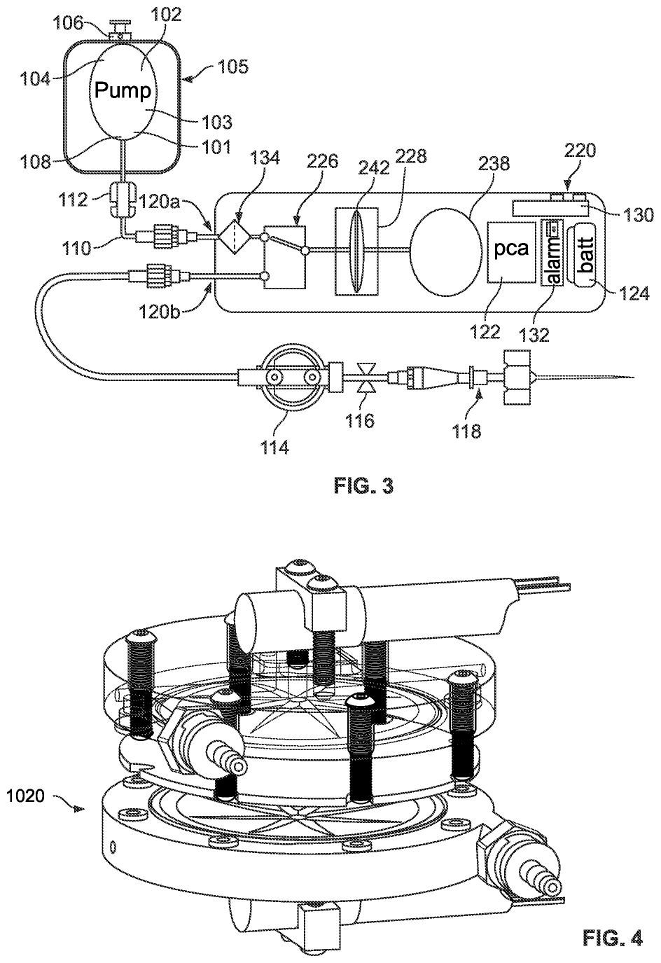

Turning to FIG. 3, an alternative flow rate monitor 220 is provided that includes many similar components of the flow rate monitor 120 and thus includes similar suffixes. These similar features will not be discussed in further detail. The flow rate monitor 220 differs from the flow rate monitor 120 in that it includes a single three-way valve 226 and a pressure vessel 238 that exerts an opposing pressure on the diaphragm 242 to expel the drug 101. Turning to FIG. 4, an example flow rate monitor 1020 may be provided with an integrated manifold therein, thereby eliminating the need for external routing. The flow rate monitor 1020 could be embodied with either the monitor 120 in FIG. 1, the monitor 220 in FIG. 3, or another suitable flow rate monitor such a flow rate monitor known in the art. Such a design is compact and provides for increased repeatability and performance due to only one side of the diaphragm being used (filled and dispensed), so each dispense cycle has a repeatable volume as compared to using both sides of the diaphragm where each side may have minor differences in volume that can impact repeatability.

The end of travel sensor 146 may be used to determine if under delivery of the drug 101 may be sufficient. For example, a pressure differential may be present if the delivery cycle was successful, or equal input/output pressures may be expected if the cycle was unsuccessful. Accordingly, a differential pressure sensor may be positioned on the inlet/outlet lines that determine whether to reject an “increment” to the cycle count that updates the delivered volume.

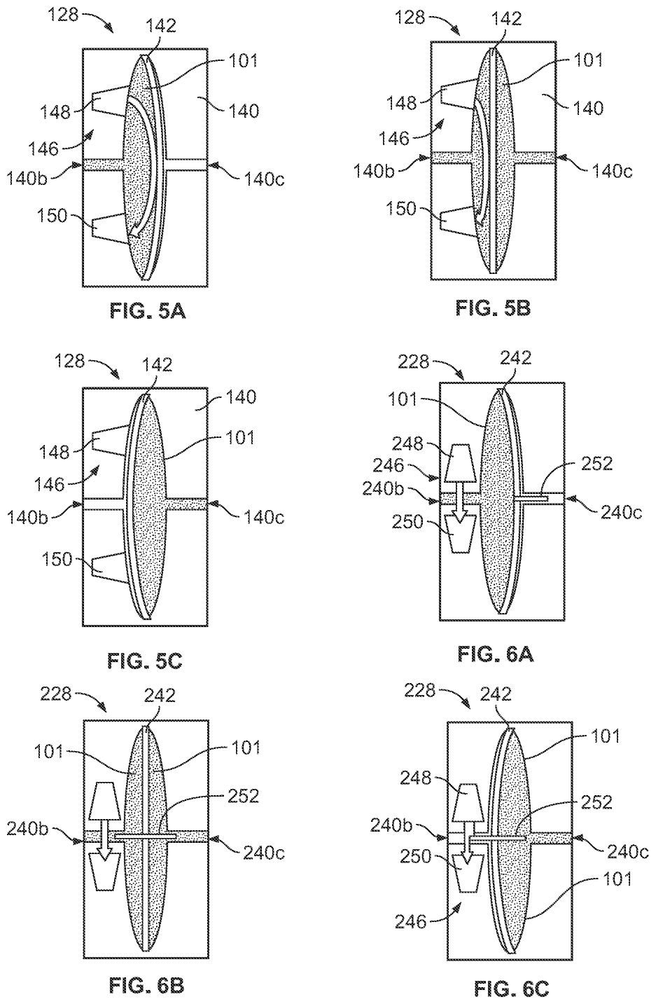

Turning to FIGS. 5a-5c, a diaphragm assembly 128 having a first example end of travel sensor 146 is provided. The end of travel sensor 146 is in the form of an optical sensor that includes a transmitter 148 (e.g., a light emitting diode (“LED”)) that transmits an optical signal into the internal volume 140a of the reservoir 140. The optical sensor further includes a light sensor 150 that receives the input light signal. The internal volume 140a and/or the diaphragm 142 act as a “light pipe” that channels the light towards the light sensor 150. As illustrated in FIG. 5b, during movement of the diaphragm 142 towards the first side port 140b, the light output received by the light sensor 150 decreases, and in FIG. 5c, when the diaphragm 142 is positioned at or near a sidewall of the reservoir 140, the light sensor 150 ceases to receive light emitted by the transmitter 148. Accordingly, the light sensor 150 may use this lack of light input as an indication that the diaphragm 142 has reached its end of travel, and will transmit a signal to the digital controller 122.

Turning to FIGS. 6a-6c, a diaphragm assembly 228 having a second example end of travel sensor 246 is provided. The diaphragm assembly 228 includes a number of similar components as the diaphragm assembly 128, and thus FIGS. 6a-6c include reference to components having reference numerals with identical suffixes as the diaphragm assembly 128. The end of travel sensor 246 is in the form of a corresponding transmitter 248 and light sensor 250 that are disposed at or near the first side port 240b or the second side port 140c of the reservoir 240. The end of travel sensor 246 additionally includes a finger 252 coupled to the diaphragm 242 that moves within the first or second side port. As the finger 252 enters the first side port 240b, it interrupts the beam of light transmitted by the light sensor 250, thus indicating an end of travel.

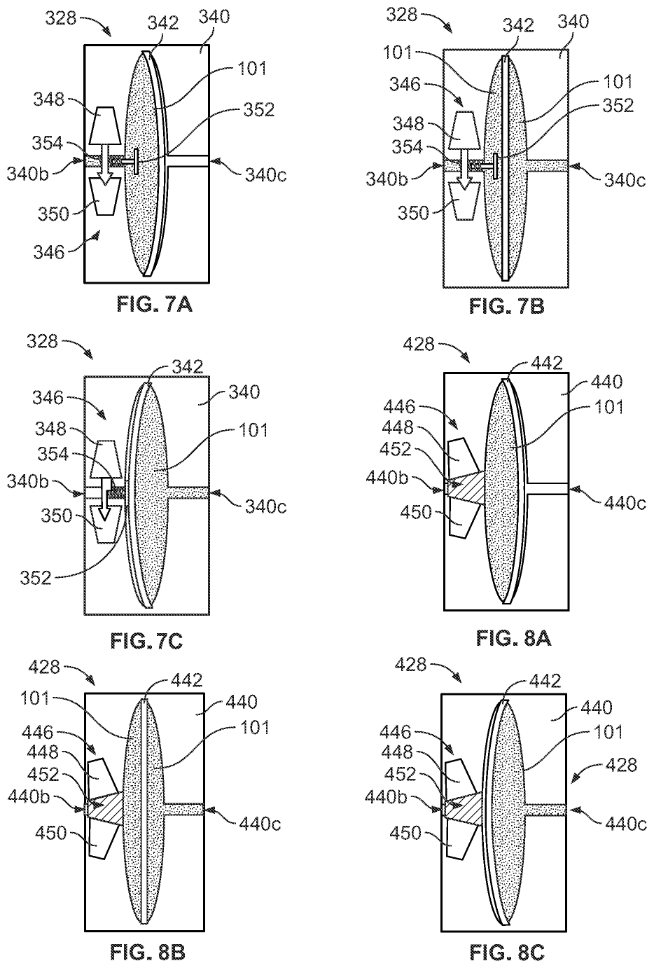

Turning to FIGS. 7a-7c, a diaphragm assembly 328 having a third example end of travel sensor 346 is provided. The diaphragm assembly 328 includes a number of similar components as the diaphragm assemblies 128 and 228, and thus FIGS. 7a-7c include reference to components having reference numerals with identical suffixes as the diaphragm assemblies 128 and 228. The end of travel sensor 346 is in the form of a push-button mechanism having a platform finger member 352 and a spring member 354 disposed within or near the first or second side port 340b, 340c. In this example, the diaphragm 342 pushes the platform finger member 352 into the first side port 340b until it interrupts the beam of light between the transmitter 348 and the light sensor 350, thus indicating an end of travel. Such a configuration provides robustness against varying assembly tolerances.

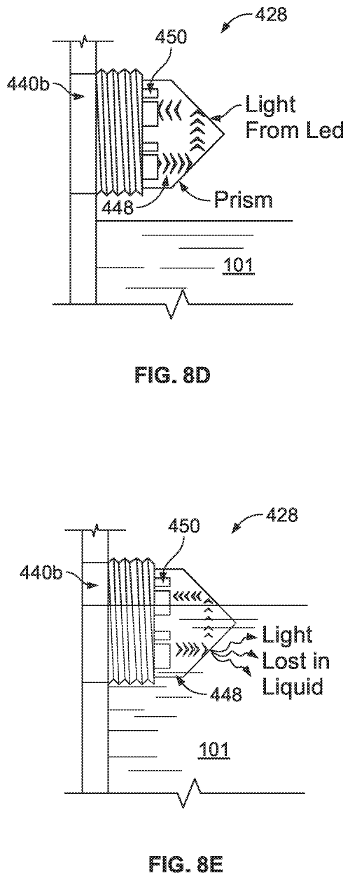

Turning to FIGS. 8a-8e, a diaphragm assembly 428 having a fourth example end of travel sensor 446 is provided. The diaphragm assembly 428 includes a number of similar components as the diaphragm assemblies 128, 228, and 328, and thus FIGS. 8a-8e include reference to components having reference numerals with identical suffixes as the diaphragm assemblies 128, 228, and 328. The end of travel sensor 446 is in the form of a prism or light pipe that changes color when in contact with a membrane (FIGS. 8a-8c) and/or may exhibit a change in refractive properties under certain circumstances (FIGS. 8d and 8e). For example, as illustrated in FIGS. 8a and 8b, the light 452 emitted by the transmitter 448 may be a first color (as indicated by cross-hatching in a first direction) when the diaphragm is not positioned at or near the sidewall of the reservoir 440 closest to the first side port 440b. However, as illustrated in FIG. 8c, upon the diaphragm 442 being positioned near the sidewall of the reservoir 440, the light 452 emitted by the transmitter 448 may change colors (as indicated by cross-hatching in a second direction). The light sensor 450 may sense this change in color thus indicating an end of travel. The end of travel sensor 446 may additionally include a colored dot positioned along the membrane 442 to assist in signaling a change in color. As illustrated in FIGS. 8d and 8e, material contact may cause a reflection loss or change in color that can be sensed by the light sensor 450. The light emitted by the transmitter 448 may exhibit a change in refractive properties upon being at least partially immersed in the drug 101, thus causing the sensor 450 to sense this change in characteristic as an indication as an end of travel of the diaphragm 442. Other examples are possible.

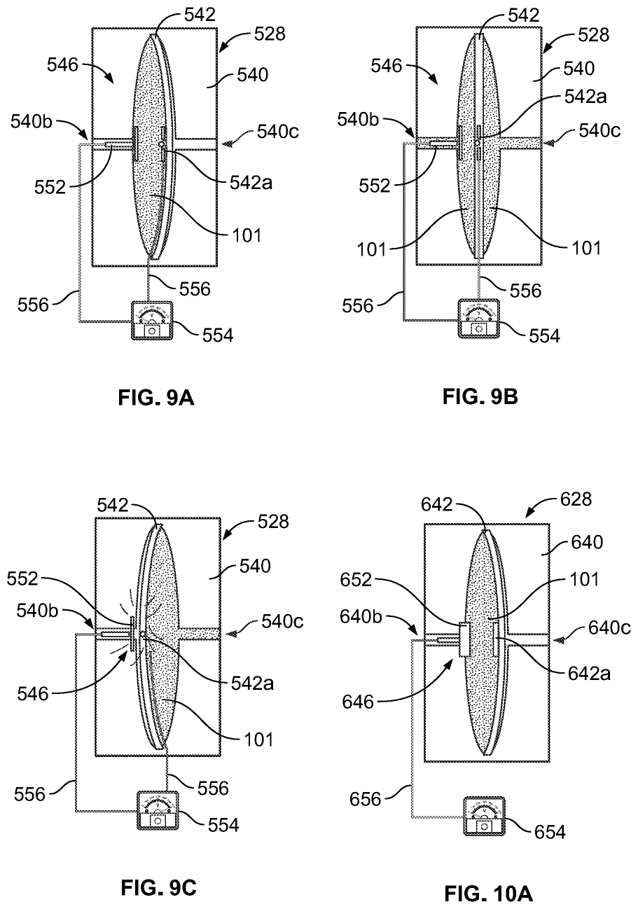

Turning to FIGS. 9a-9c, a diaphragm assembly 528 having a fifth example end of travel sensor 546 is provided. The diaphragm assembly 528 includes a number of similar components as the diaphragm assemblies 128, 228, 328, and 428, and thus FIGS. 9a-9c include reference to components having reference numerals with identical suffixes as the diaphragm assemblies 128, 228, 328, and 428. The end of travel sensor 546 is in the form of an electrical circuit that uses direct electrical contact between the membrane 542 and a sensing pad 552. The membrane 542 and the sensing pad 552 are coupled to a meter 554 or similar device via wires or cables 556. In some examples and as illustrated in FIGS. 9a-9c, the diaphragm 542 may additionally have a sensing pad or contact 542a that transmits the electrical signal to the sensing pad 552. Other examples are possible.

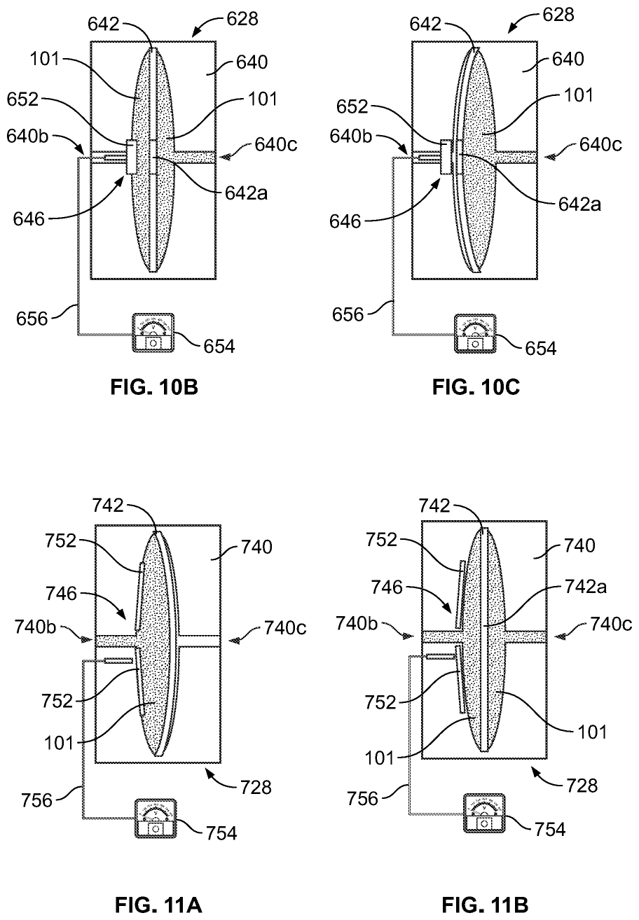

Turning to FIGS. 10a-10c, a diaphragm assembly 628 having a sixth example end of travel sensor 646 is provided. The diaphragm assembly 628 includes a number of similar components as the diaphragm assemblies 128, 228, 328, 428, and 528, and thus FIGS. 10a-10c include reference to components having reference numerals with identical suffixes as the diaphragm assemblies 128, 228, 328, 428, and 528. The end of travel sensor 646 is in the form of a magnetic Hall Effect sensor 652 and a magnetized membrane 642 or portion of the membrane 642a to sense membrane proximity. The sensor 652 is coupled to a meter 654 or similar device via wires or cables 656. Other examples are possible.

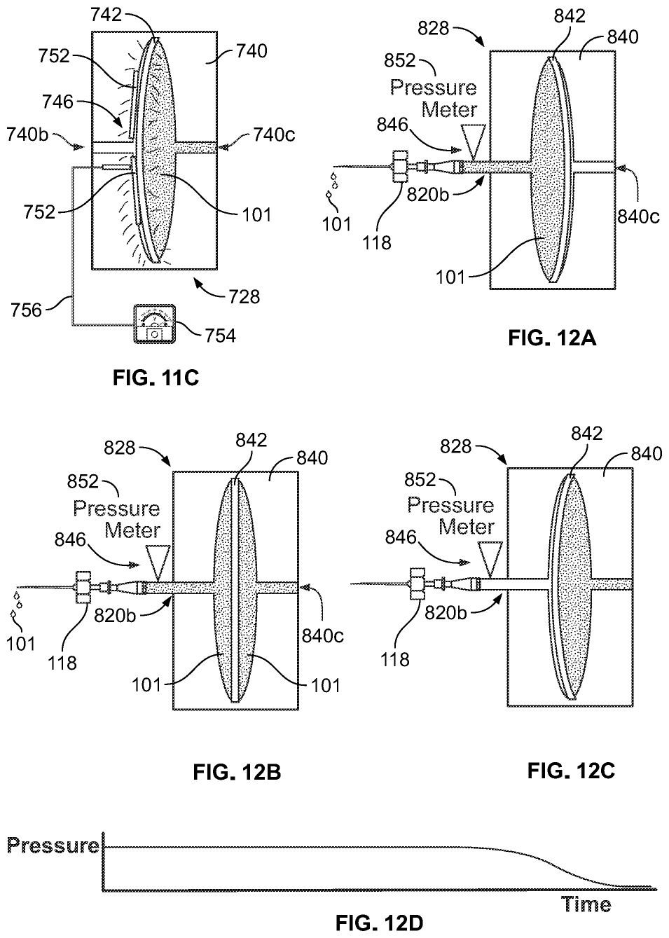

Turning to FIGS. 11a-11c, a diaphragm assembly 728 having a seventh example end of travel sensor 746 is provided. The diaphragm assembly 728 includes a number of similar components as the diaphragm assemblies 128, 228, 328, 428, 528, and 628, and thus FIGS. 11a-11c include reference to components having reference numerals with identical suffixes as the diaphragm assemblies 128, 228, 328, 428, 528, and 628. The end of travel sensor 746 is in the form of a capacitive sensor 752 that cooperates with a capacitive membrane 742 or portion of the membrane 742a to sense a change in capacitance that indicates membrane proximity. The sensor 752 is coupled to a meter 754 or similar device via wires or cables 756. Other examples are possible.

Turning to FIGS. 12a-12d, a diaphragm assembly 828 having an eighth example end of travel sensor 846 is provided. The diaphragm assembly 828 includes a number of similar components as the diaphragm assemblies 128, 228, 328, 428, 528, 628, and 728, and thus FIGS. 12a-12c include reference to components having reference numerals with identical suffixes as the diaphragm assemblies 128, 228, 328, 428, 528, 628 and 728. The end of travel sensor 846 is in the form of a pressure sensor or monitor 852 that senses the end of travel of the diaphragm 842, and additionally may sense the presence of occlusions within the fluid flow path. Specifically, the pressure monitor is positioned at or near the outlet 820b of the flow rate monitor 820 (or at any location downstream of the fluid valve) and monitors the pressure of the drug. As illustrated in FIG. 12d, when the sensor 852 measures a reduction in pressure, the diaphragm 842 has reached its end of travel. Other examples are possible.

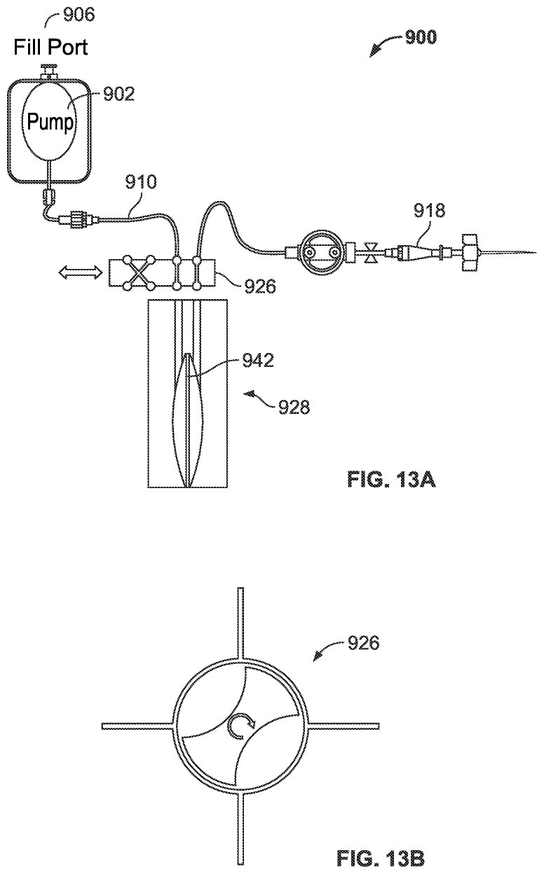

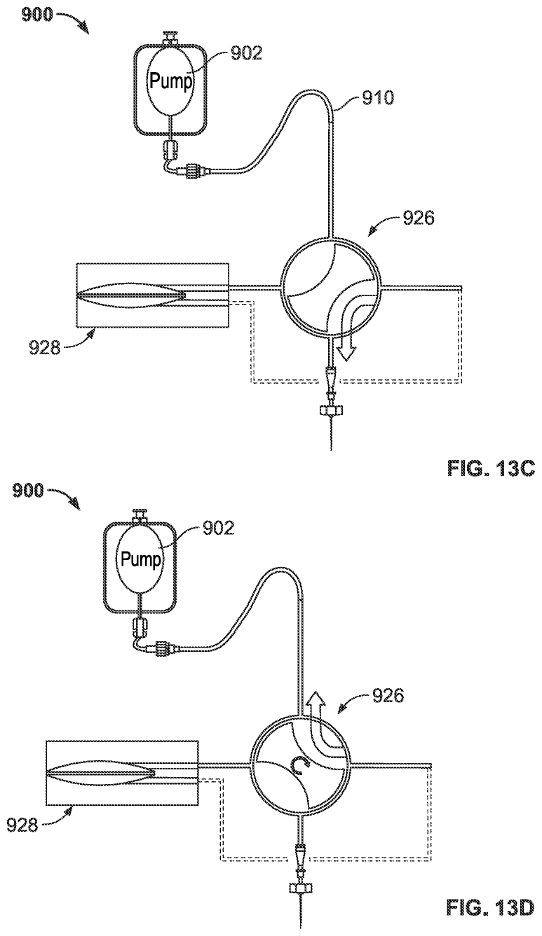

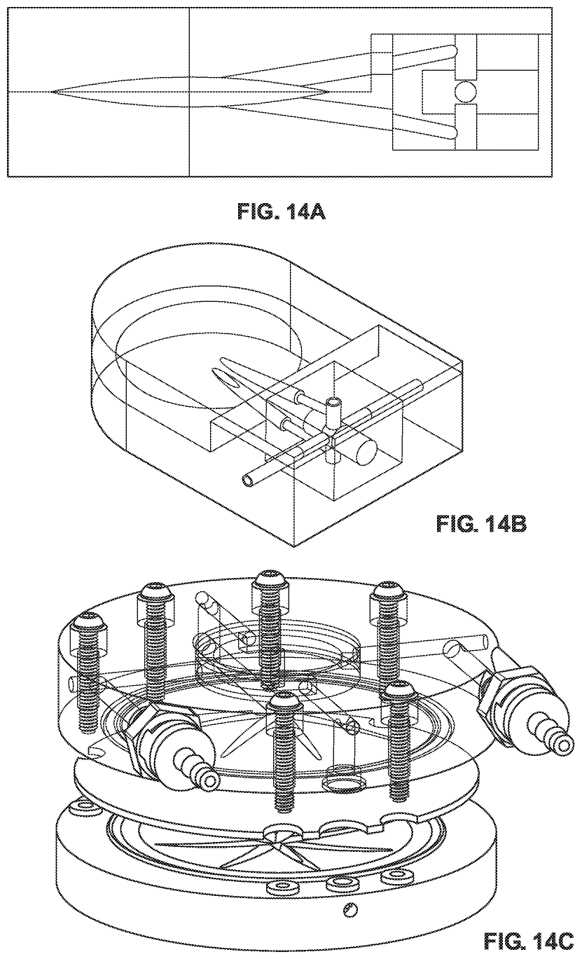

Turning to FIGS. 13a-13d, a system 900 is provided that has an alternative fluid valve 926. The system 900 includes a number of similar components as the embodiments illustrated in FIGS. 1-12d, and thus FIGS. 13a-13d include reference to components having reference numerals with identical suffixes as the embodiments illustrated in FIGS. 1-12d. The system 900 includes a four-way fluid valve 926 that allows for selective fluid paths between the pump 902 and the diaphragm assembly 928 (FIG. 13c), and the pump 902 directly to the needle 918 (FIG. 13d). As illustrated in FIGS. 14a-14c, a four-way valve is provided that is integral and requires no large solenoids. This four-way valve rotates approximately 90° for each successive filling or emptying of the reservoir 140, thereby offering free-flow prevention, low power requirements, and wide flow path tolerances.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNa mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery system comprising:
a delivery container including a container body adapted to accommodate a drug therein, an inlet port and an outlet port, the container body being constructed from a resilient material such that the container body is adapted to exert an urging force on the drug to expel the drug from the outlet port;
a supply line operably coupled to the outlet port to deliver the drug to a user; and
a flow rate monitor operably coupled to at least one of the delivery container or the supply line, the flow rate monitor comprising:
a digital controller,
a fluid valve operably coupled to the digital controller,
a diaphragm assembly in fluid communication with the fluid valve and being operably coupled to the digital controller, wherein the fluid valve, the diaphragm assembly, and the digital controller cooperate to regulate a flow rate of the drug.

2. The drug delivery system of claim 1, wherein the digital controller is adapted to cause the flow rate monitor to actuate the fluid valve.

3. The drug delivery system of claim 1, wherein the fluid valve comprises:
a primary three-way valve including a primary valve inlet, a first primary valve outlet, and a second primary valve outlet; and
a secondary three-way valve including a first secondary valve inlet, a second secondary valve inlet, and a secondary valve outlet;
wherein the primary three-way valve is ganged to the secondary three-way valve.

4. The drug delivery system of claim 1, wherein the fluid valve comprises a four-way valve.

5. The drug delivery system of claim 3, wherein the diaphragm assembly comprises:
a reservoir defining an internal volume, a first side port, and a second side port; and
a diaphragm disposed within the internal volume of the reservoir between the first side port and the second side port to define a first cavity and a second cavity.

6. The drug delivery system of claim 5, wherein the diaphragm is constructed from a resilient material.

7. The drug delivery system of claim 5, further comprising at least one end of travel sensor configured to sense at least one directional limit of the diaphragm.

8. The drug delivery system of claim 7, wherein the at least one end of travel sensor comprises an optical sensor.

9. The drug delivery system of claim 7, wherein the at least one end of travel sensor comprises at least one of an electrical contact sensor or a capacitive sensor.

10. The drug delivery system of claim 7, wherein the at least one end of travel sensor comprises a hall effect sensor.

11. The drug delivery system of claim 7, wherein the at least one end of travel sensor comprises a pressure monitor.

12. The drug delivery system of claim 1, wherein the flow rate monitor further comprises: 1) an interface coupled to the digital controller to receive at least one input; and 2) a display coupled to the digital controller.

13. The drug delivery system of claim 1, further comprising an alarm operably coupled to the digital controller.

14. The drug delivery system of claim 1, further comprising at least one of an air trap, a filter, or a flow restrictor, or a fluid path compliance member downstream of the flow controller.

15. A drug delivery system comprising:
a delivery container including a container body adapted to accommodate a drug therein, an inlet port and an outlet port, the container body receiving a driving force that causes the container body to exert an urging force on the drug to expel the drug from the outlet port;
a supply line operably coupled to the outlet port to deliver the drug to a user; and
a flow rate monitor operably coupled to at least one of the delivery container or the supply line, the flow rate monitor comprising:
a digital controller,
a fluid valve operably coupled to the digital controller,
a diaphragm assembly in fluid communication with the fluid valve and being operably coupled to the digital controller, wherein the fluid valve, the diaphragm assembly, and the digital controller cooperate to regulate a flow rate of the drug.

16. The drug delivery system of claim 15, wherein the driving force is generated by at least one of a spring or a non-resilient surface that translates linearly in a cylinder under pressure from a spring.

17. The drug delivery system of claim 15, wherein the fluid valve comprises:
a primary three-way valve including a primary valve inlet, a first primary valve outlet, and a second primary valve outlet; and
a secondary three-way valve including a first secondary valve inlet, a second secondary valve inlet, and a secondary valve outlet;
wherein the primary three-way valve is ganged to the secondary three-way valve.

18. The drug delivery system of claim 15, wherein the fluid valve comprises a four-way valve.

19. The drug delivery system of claim 17, wherein the diaphragm assembly comprises:
a reservoir defining an internal volume, a first side port, and a second side port; and
a diaphragm disposed within the internal volume of the reservoir between the first side port and the second side port to define a first cavity and a second cavity.
